(19) United States
(12) Patent Application Publication
(10) Pub. No.: US 2007/0213782 A1
(43) Pub. Date: Sep. 13, 2007

(54) Method and Apparatus for the Treatment of Sleep Apnea and Snoring

(76) Inventor: David Peter Shaw, Christchurch (NZ)

Correspondence Address: WELLS ST. JOHN P.S., 601 W. FIRST AVENUE, SUITE 1300, SPOKANE, WA, 99201, US

(21) Appl. No.: 10/593,437

(22) PCT Filed: Mar. 18, 2005
(86) PCT No.: PCT/NZ05/00050

§ 371 Date: Sep. 18, 2006

(30) Foreign Application Priority Data
Mar. 18, 2004 (NZ) 531829

(51) Int. Cl. A61N 1/00

(52) U.S. Cl. 607/42

(57) ABSTRACT

A method of treating sleep apnoea and/or snoring in a patient, which includes the steps of providing apparatus for electrically stimulating one or more afferent fibres of a nerve (2), positioning said apparatus (12, 13) on or in close proximity to said nerve; and activating said apparatus to stimulate one or ore afferent fibres of said nerve (2).

### TECHNICAL FIELD

The present invention relates to a method and apparatus for the treatment of sleep apnoea and snoring.

### BACKGROUND ART

Sleep apnoea is a condition of failing to breathe during sleep. The breathing process is complex and requires the coordination of many muscles within the body:—the diaphragm, the intercostal muscles, and the chest wall muscles. In addition, the muscles associated with the mouth and the upper airway have to be coordinated so that there is no obstruction to the airflow.

Failure to breathe may be due to:
a) A reduction in the tone of the pharyngeal muscles, so that these muscles are not retracted to allow the unimpeded entry/exit of air, or obstruction to the passage of air caused by a large tonsils or adenoids, or by an abnormally large tongue or small jaw (obstructive sleep apnoea);
b) The diaphragm and chest muscles temporarily ceasing to work, probably as result of a disturbance in the brain's control of breathing (central sleep apnoea);
c) A combination of (a) and (b).

Sleep apnoea affects approximately 2% of the population. Severe sleep apnoea is potentially serious:—it is known to be a factor in high blood pressure, heart failure, heart attacks and strokes. Further, a sufferer from sleep apnoea may be excessively sleepy during the day, and may fall asleep while driving or trying to work and/or may suffer from poor memory and concentration.

Sleep apnoea caused by structural problems such as large tonsils or adenoids or by an abnormally large tongue or small jaw may be treated surgically. Sleep apnoea caused by non-structural problems often is treated by continuous positive airway pressure (CPAP):—compressed air is forced into the sleeper's airway via a mask worn over the nose. Since this treatment has to be given for the full sleeping period, every night, and the CPAP device is noisy and the mask is uncomfortable, the treatment is unpleasant both for the patient and his or her immediate family.

Snoring often is associated with obstructive sleep apnoea, but may occur independently of sleep apnoea. Although snoring is a minor problem compared to sleep apnoea, a persistent loud snorer can cause severe sleep disruption in any one trying to sleep in the same room, and there are no reliable treatments for snoring unless the snoring is caused by a structural problem (see above) which can be treated surgically.

### DISCLOSURE OF INVENTION

It is therefore an object of the present invention to provide a method and apparatus for the treatment of sleep apnoea and snoring which overcomes the drawbacks of the present non-surgical treatments and provides a viable alternative to present treatments.

The present invention provides a method of treating sleep apnoea and/or snoring which includes the steps of:—
a) providing apparatus for electrically stimulating one or more afferent fibres of a nerve;
b) positioning said apparatus on or in or in close proximity to said nerve;
c) activating said apparatus to stimulate said one or more afferent fibres of said nerve.

Preferably, it is the respiratory centre of the brain which is stimulated by the electrical stimulation of one or more afferent fibres of a nerve; most preferably the afferent fibres are the afferent fibres of the phrenic nerve.

In one preferred embodiment of the invention, the afferent fibres stimulated by the apparatus are the large mylinated afferent fibres having a diameter in the range of 12 to 20 micrometers. Preferably, these fibres are proprioceptor fibres, because stimulation of these fibres does not reach the cortex, and thus does not reach the consciousness and wake the patient up. It follows that it is possible to treat the sleep apnoea or snoring using this method without interrupting the sleep of the patient.

The apparatus may include a sensor provided within, or on, or adjacent the patient; the sensor is adapted to detect the condition to be treated and is arranged to activate the apparatus upon detecting that condition.

The type of sensor used naturally depends upon the condition to be treated:—in the case of snoring, an audio sensor or vibration sensor set up to detect noises on the normal wavelength of snoring noise could be used.

In the case of sleep apnoea, the sensor would be one capable of sensing the failure of normal respiration:—that is, either the cessation of breathing (as in central sleep apnoea) or the irregular rhythm which occurs when there is attempted breathing but with no airflow (as in obstructive sleep apnoea). Suitable sensors may include external or internal sensors; for example, external mechanical or electrical devices to measure chest wall movement, (e.g. thoracic impedance) or internal devices for transvenous sensing of respiration (such as impedance sensing) or for detecting physical deformation, (e.g. transvenous fibre optics), or for detecting sound or vibration.

### BRIEF DESCRIPTION OF DRAWING

By way of example only, preferred embodiments of the present invention as applied to the treatment of sleep apnoea is described in detail with reference to the accompanying drawings, in which:—

FIGS. 1 and 2 show diagrammatic cross-sections through the chest of a patient with first and second embodiments, respectively, of the present invention; and

FIG. 3 shows a sketch front view of the neck and chest of a patient fitted with a third embodiment of the present invention.

### BEST MODE FOR CARRYING OUT THE INVENTION

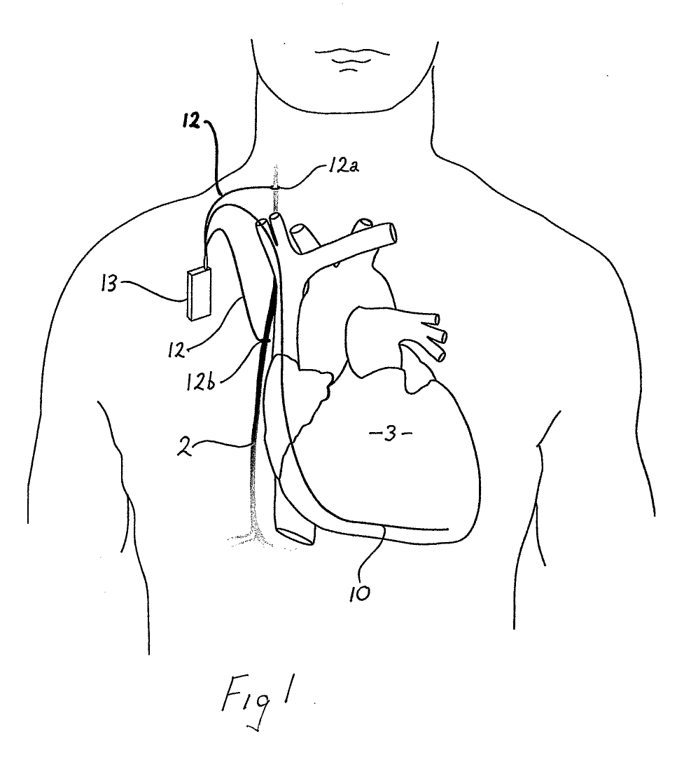

In a preferred embodiment of the present invention, as shown in FIG. 1, the treatment of sleep apnoea using the method and apparatus of the present invention is based on the premise that the afferent fibres of the phrenic nerve are integrated into the coordination of breathing.

The human body is designed so that every muscle or set of muscles has controlling nerves, and virtually every nerve has both afferent and efferent fibres. The afferent fibres feed back from the muscles to the brain, and the efferent fibres control the reaction of the muscles in response to the stimuli being processed by the brain. In breathing, the efferent fibres control the muscles of the diaphragm, the intercostal muscles, and the muscles of the chest wall; these coordinate with the muscles of the upper airways (the pharyngeal muscles) which appropriately constrict or relax. The afferent fibres feed back to the respiratory centre in the brain information on muscle position and chest wall position. The respiratory centre must also integrate a timing mechanism for the frequency of breaths, which includes monitoring the activity level and the carbon dioxide and oxygen levels so that respiration is increased or decreased as appropriate.

The phrenic nerve contains efferent motor fibres that innervate the muscle of the diaphragm, additionally there are afferent sensory fibres that provide sensation to the diaphragmatic and mediastinal pleura plus proprioceptor fibres that transmit information to the brain concerning muscle movement and position. Unlike some afferent fibres, (e.g. pain) these do not reach the cortex, thus do not reach consciousness. Additionally, they are large diameter mylinated fibres (diameter in the range 12 to 20 micrometers) that have the lowest threshold for electrical stimulation, thus it is possible to stimulate these afferent fibres while not stimulating others.

Input from proprioceptor fibres is one of the many parameters that the respiratory centre in the brain weighs prior to deciding to initiate and co-ordinate a breath.

The phrenic nerve is one of the nerves associated with the muscles used in breathing. It is believed that electrical stimulation of the afferent fibres of the phrenic nerve during an episode of sleep apnoea will cause the respiratory centre to realise that a breath should be taken, and thus initiate the coordination of breathing, including increasing tone in the pharyngeal muscles to open the airway.

Particular reference is made to the phrenic nerve because the technology for stimulation of the phrenic nerve is already developed (for cardiomyoplasty, phrenic nerve stimulation in spinal patients, and spinal stimulation) and because the phrenic nerve is easy to identify, for fitting the apparatus as described below. However, it must be emphasised that the method and apparatus of the present invention are not limited to use with the phrenic nerve:—other nerves associated with the breathing process could be used instead.

Referring to the FIG. 1 of the drawing, part of the phrenic nerve 2 is shown, extending alongside the heart 3.

A sensor in the form of a transvenous lead 10 is inserted down the superior vena cava of the patient into the right ventricle. A second lead 12 is attached to the phrenic nerve; two possible attachment points 12a/12b are shown. The other ends of the leads 11 and 12 are connected to a small generator/computer 13 which could be mounted externally on the patient, but which preferably is mounted in a subcutaneous pocket in the patient in a similar manner to a heart pacemaker, as shown.

In the event that the sensor 10 detects either the failure of respiration, or those movements which suggest obstruction of the airway, then the sensor 10 activates the generator/computer 13 which in turn creates an electrical stimulation of the phrenic nerve via the lead 12. The stimulation of the phrenic nerve causes a contraction of the diaphragm and stimulation of the proprioceptor afferent phrenic nerve fibres to the respiratory centre. This causes the respiratory centre to initiate a coordinated breath action, which includes increasing the tone of the pharyngeal muscles and thus opening the airway.

It will be appreciated that, since stimulation of the phrenic nerve will in turn stimulate the respiratory centre, and will increase the tone of the pharyngeal muscles, this technique will be effective in treating either or both central sleep apnoea and obstructive sleep apnoea. The degree of stimulation of the phrenic nerve is selected according to known data available on nerve stimulation such that only the proprioceptor fibres are stimulated:—as noted above, these fibres have the lowest threshold for electrical stimulation and it therefore is possible to stimulate these fibres whilst not stimulating other fibres of the nerve which would reach the consciousness and/or cause pain.

As described above, other types of the sensor may be used.

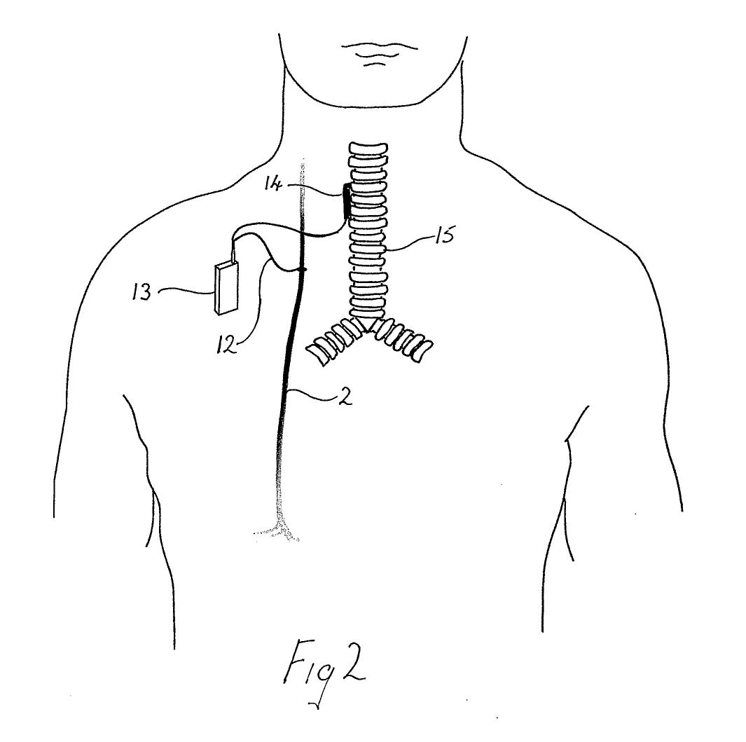

FIG. 2 shows the use of a vibration sensor 14, for detecting the abnormal vibration caused by snoring. The sensor 14 is located adjacent the trachea 15 of the patient, and is connected to a generator/computer 13 which also is connected to a lead 12, attached to the phrenic nerve 2. If the vibration sensor is activated by the vibration of snoring, the generator/computer 13 causes electrical stimulation of the phrenic nerve via the lead 12, as described with reference to FIG. 1.

For some applications, it is envisaged that a sensor will not be necessary:—if the patient is one who always suffers from severe sleep apnoea every night, then the apparatus may be provided without a sensor and simply set to provide a continuous or a periodic stimulation as required.

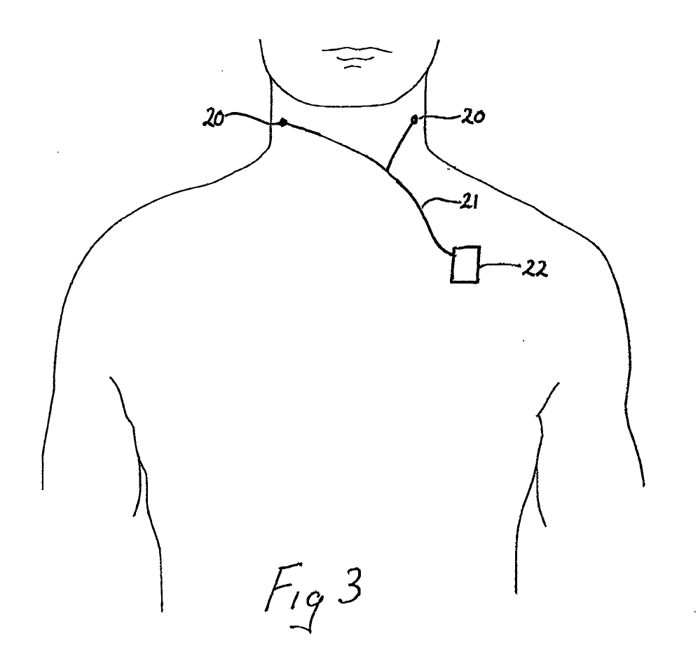

A third embodiment of the invention is shown in FIG. 3. In this embodiment, the apparatus is completely external and consists of a pair of stick on electrodes 20 which are connected by short leads 21 to a generator/computer 22 which is clipped or otherwise secured to the clothing of the patient by any suitable means. The electrodes 20 are stuck onto the neck in the region of the phrenic nerve, which is easily located because it is adjacent the carotid artery. The electrodes 20 may be stuck on opposite sides of the neck, as shown, or may be stuck on one side of the neck spaced apart vertically.

The apparatus may be set to operate automatically or may also include a sensor, e.g. a vibration sensor to detect snoring or a motion sensor to detect the absence of airflow.

It will be appreciated that this apparatus is completely non-invasive and is relatively easy and pleasant for a patient to use. Also, the fact that the apparatus can be fitted and removed easily means the apparatus is very suitable for testing a patient to see whether this type of treatment is suitable for that particular patient.

The amount of stimulation required to achieve the desired effect varies depending upon the nerve being stimulated; techniques of accurate stimulation of nerves to a pre-determined degree are well established.

1-2. (canceled)

3. A method of treating sleep apnoea and/or snoring in a patient which includes the steps of:
a) providing apparatus for electrically stimulating one or more afferent fibres of the phrenic nerve;
b) positioning said apparatus on or in close proximity to said nerve;
c) activating said apparatus to stimulate said one or more afferent fibres.

4. A method of treating sleep apnoea and/or snoring in a patient which includes the steps of:
a) providing apparatus for stimulating the respiratory centre by electrically stimulating one or more afferent fibres of the phrenic nerve;
b) positioning said apparatus on or in close proximity to said nerve;
c) activating said apparatus to stimulate said one or more afferent fibres of the phrenic nerve and hence stimulate the respiratory centre.

5. A method of treating sleep apnoea and/or snoring in a patient which includes the steps of:
a) providing apparatus for stimulating the respiratory centre by electrically stimulating the proprioceptor fibres of the phrenic nerve;
b) positioning said apparatus on or in close proximity to said nerve;
c) activating said apparatus to stimulate said fibres and hence stimulate the respiratory centre.

6. The method as claimed in any one of claims 3-5, wherein the afferent fibres are the large mylinated afferent fibres having a diameter in the range of 12-20 micrometers.

7. The method as claimed in any one of claims 3-5, wherein said apparatus is located wholly or partially internally of the patient.

8. The method as claimed in any one of claims 3-5, wherein said apparatus is located externally upon the patient, and said nerve is stimulated transcutaneously.

9. The method as claimed in any one of claims 3-5, further including the step of providing a sensor in, on, or adjacent the patient; said sensor is adapted to detect the condition to be treated and is arranged to activate said apparatus upon detecting said condition.

10. The method as claimed in claim 8, wherein the sensor is selected from the group consisting of:
a vibration sensor;
a transvenous lead;
a sound sensor;
a thoracic impedence sensor

11. The method as claimed in claim 6, wherein said apparatus is located wholly or partially internally of the patient.

12. The method as claimed in claim 6, wherein said apparatus is located externally upon the patient, and said nerve is stimulated transcutaneously.
